(19) United States
(12) Patent Application Publication
(10) Pub. No.: US 2005/0271751 A1
(43) Pub. Date: Dec. 8, 2005

(54) Active principle which is capable of inducing the conversion of inactive TGFb-latent into active TGFb

(75) Inventors: Eric Perrier, Les Cotes D'Arey (FR); Cecile Altobelli, Jons (FR); Anne Clement, Paris (FR); Corrinne Reymermier, Charly (FR); Nabil Abdul Malak, Caluire (FR)

Correspondence Address: MERCHANT & GOULD PC, P.O. BOX 2903, MINNEAPOLIS, MN, 55402-0903, US

(73) Assignee: LABORATOIRE DE DERMOCOSMETIQUE, Paris (FR)

(21) Appl. No.: 10/966,168

(22) Filed: Oct. 4, 2004

(30) Foreign Application Priority Data
Jun. 4, 2004 (FR) 04 06047

(51) Int. Cl. A61K 35/78

(52) U.S. Cl. 424/727; 424/750; 424/757; 424/769; 424/773

(57) ABSTRACT

The invention relates to active principles which are capable of inducing the conversion of inactive TGFb-Latent into active TGFb. The invention relates in particular to the use of such an extract for the manufacture of a composition for increasing the concentration of active TGFb1 in the skin, notably in the dermis. The present invention also relates to the uses which are derived from said use, as well as to cosmetic or pharmaceutical compositions which comprise said extract.

The invention relates to natural extracts which convert Latent Transforming Growth Factor beta 1 (TGFb1-L) into Transforming Growth Factor beta 1 (TGFb1) which is the “active” form of TGFb1-L, and to their uses in cosmetics, dermopharmaceuticals and pharmaceuticals, notably for increasing the concentration of TGFb1 in the skin, notably in the dermis.

### STATE OF THE ART

Amongst the growth factors and the cytokines, TGFb1, which is secreted in the skin by numerous cells, including the keratinocytes, the fibroblasts, the leukocytes and the platelets, is one of the most efficient regulators of healing, via its significant properties of modifying cell metabolism and the re-modelling of the extracellular matrix (Rousselle P et al., Ed. Médias Flash (1998); Melissopoulos A et al., Ed. Médicales Internationales, (1998)).

In man, four isoforms have been identified to date in the TGF-beta family. They are transcribed and translated from genes which are totally distinct and which are present on different chromosomes. TGF-beta 1 is positioned on the 19q13 chromosome, whereas TGF-beta 2 is positioned on the 1q41 chromosome, TGF-beta 3 on the 14q24 chromosome, and TGF-beta 4 on the 1q42.1 chromosome.

TGF-beta-1 is secreted and stored in biologically inactive form, called “latent” form, and must be “activated” so as to acquire its biological effectiveness.

Latent TGFb1 is stored in the extracellular matrix and represents, in the aged subject, a reservoir which is not used.

In vivo, various factors are capable of inducing the activation of the latent TGFb1:
glucosidases (endoglycosidase-F, neuraminidase, N-glycanase),
sialidases which are secreted in the macrophages,
serine proteases (plasmin, cathepsin D and stromelysin (MMP3),
thrombospondin-1, an adhesion protein which binds to the surface of the cells but also to the extracellular matrix, is the main physiological regulator of the activation of latent TGFb1.

Active TGFb1 is considered to be the most important multifunctional growth actor of the development and of the homeostasis of the skin, which enables, notably in the cells, the induction of the proliferation of the fibroblasts, their chemoattraction, the stimulation of the neovascularisation, the differentiation of the fibroblasts into myofibroblasts, and the regulation of the growth of the fibroblasts. It also enables, in the extracellular matrix, increasing the synthesis of the collagens, decreasing the collagenases and increasing the synthesis of the protease inhibitors (TIMP), increasing the expression of the isoforms of fibronectin and the synthesis of the fibronectin receptors, increasing the synthesis of elastin. It is however also involved in increasing the synthesis of the proteoglycans and hyaluronic acid.

A decrease in the concentration of active TGFb1 in the dermis is observed, whereas the fibroblasts maintain their capacity to respond to a stimulation by the active TGFb1.

This decrease of concentration of active TGFb1 during ageing induces a decrease of the proliferation of the fibroblasts, a decrease of the synthesis of the constituents of the extracellular matrix (ECM) and inhibits the destructive activity of the extracellular proteins of the ECM.

Methods of activation have been proposed amongst the physical treatments (temperature), chemical treatments (acidic pHs) or enzymatic activation.

However, these activation methods are:

either very drastic and inapplicable to rational uses of cosmetic formulation (example: high temperatures which cannot be used, or very acidic pHs which cannot be envisaged for cosmetic applications),

or make use of enzymes, which are not very useful in cosmetics, on the one hand since they can induce allergenic reactions on the skin, and on the other hand since their very high molecular weights do not enable their penetration to be obtained into the deep layers of the skin, including the dermis, a zone in which the TGF beta is present in its inactive form.

These methods do not enable a cosmetically, dermatologically, or pharmaceutically acceptable natural active principle to be provided.

Only the FR 2,810,323 patent application describes a molecule which enables the latent form of TGFb1 to be activated. This molecule is a derivative of the tripeptide lysine-phenylalanine-lysine (Lys-Phe-Lys), which sequences are present in thrombospondin-1. However, in order to be cosmetologically active, this tripeptide must be modified by grafting of an elaidyl chain by reaction of this tripeptide with elaidic acid.

This product is not natural in the sense of the present invention due to this chemical modification.

Furthermore, the person skilled in the art will think to use substances having protease activity or strong protein denaturing agents. The present invention avoids such a use which has drawbacks in the manufacture of cosmetically, or dermatologically or pharmaceutically acceptable products.

### AIM OF THE INVENTION

A main aim of the present invention is to alleviate the decrease of the concentration of active TGFb1 in the skin, and particularly in the dermis.

An aim of the present invention is to solve the novel technical problem which consists in providing natural active principles which are cosmetically, or dermatologically, or pharmaceutically acceptable, and which convert TGFb1-L into active TGFb1.

The inventors consider that the active principles are cosmetically, or dermatologically, or pharmaceutically acceptable when firstly they have no protease activity, when they can generate skin irritations or allergies, and secondly that they are not chemical substances which are known to be strong protein denaturing agents, such as strong acids of HCl type, urea, sulphur-containing reducing agents such as threitol, thioglycolic acid type, etc.

The inventors consider that active principles are natural when they are extracts from nature, such as, for example, extracts which belong to the plant kingdom (plants, algae . . . ), the mineral world, and/or parts extracted from plants (proteins, polysaccharides . . . ), from plants, from animal secretions, and/or from isolated parts of animal bodies or plants, which are eventually obtained after fermentation in the presence of bacteria. A further aim of the present invention is to solve the novel technical problem which consists in providing natural active principles which are topically acceptable and which promote the proliferation of fibroblasts.

Another aim of the present invention is to solve the novel technical problem which consists in providing natural active principles which are topically acceptable and which increase the amount of active TGFb1 and, as a consequence, which increase the synthesis of the constituents of the extracellular matrix and which inhibit the destructive activity of the extracellular proteins of the extracellular matrix. Another aim of the present invention is to solve the novel technical problem which consists in providing natural active principles which are topically acceptable and which exert an anti-wrinkle or anti-ageing effect via the mechanisms described.

The present invention notably covers providing cosmetic, dermopharmaceutical, or pharmaceutical compositions which comprise these active principles.

### SUMMARY OF THE INVENTION

The inventors have, in order to solve the various problems described above, sought which type of activation could enable the inactive TGFb1-L to be activated in vitro. Now, TGFb1 is associated with a protein LAP (latency associated protein) when it is in the inactive form (TGFb1-L) and in order to activate the TGFb1, it is necessary to cleave the bond which unites the TGFb1 to the LAP protein.

The inventors have developed a screening test which enables demonstrating the substances which activate TGFb1-L, so as to provide natural active principles which enable the concentration of active TGFb1 in the skin, notably in the dermis, to be increased.

This test demonstrated that the topically acceptable natural extracts of soya or oats, or of dwarf palm or of white mulberry tree or of Spring restharrow, or of pigeon bean or horse bean, or of tomato, or of fish roe, or of pea, or of fish, or of wheat, or of mango, or of date, or of silk, or of kiwi, or of potato, or of grapefruit, or of papaya, or of pineapple, or of passion fruit, or of scutellaria, or of maize, or of apple, or of quinoa, or of parsley, or of yucca enable TGFb1-L to be activated. This test comprises a TGFb1-L activation reaction which is preferably carried out after an incubation which is done under cosmetically and/or dermatologically acceptable conditions.

These extracts are obtained notably by any one of the extraction techniques known to the person skilled in the art.

Since these natural extracts convert TGFb1-L (TGFb1-latent) into active TGFb1 (TGFb1), they are used for the manufacture of cosmetic compositions, and/or dermopharmaceutical compositions, and/or pharmaceutical compositions, notably for increasing the concentration of TGFb1 in the skin, notably in the dermis. They are therefore also used for the properties which follow from the increase of the concentration of TGFb1 in the skin, notably in the dermis.

### DETAILED DESCRIPTION OF THE INVENTION

The invention relates principally to a natural extract, which is preferably enzymatically inactive, which converts TGFb1-L (TGFb1-latent) into active TGFb1 (TGFb1), notably by cleaving the bond which unites the TGFb1 to the protein LAP (latency associated protein), under cosmetically, dermatologically, and/or pharmaceutically acceptable conditions.

Advantageously, this plant extract is selected from an extract of soya or of oats, or of dwarf palm or of white mulberry tree or of Spring restharrow, or of pigeon bean or horse bean, or of tomato, or of fish roe, or of pea, or of fish, or of wheat, or of mango, or of date, or of silk, or of kiwi, or of potato, or of grapefruit, or of papaya, or of pineapple, or of passion fruit, or of scutellaria, or of maize, or of apple, or of quinoa, or of parsley, or of yucca, and any one of the mixtures of these extracts.

The cosmetically or dermatologically acceptable active principles of the present invention induce the conversion of inactive TGFb1-L into active TGFb1 under cosmetically or dermatologically acceptable physico-chemical conditions, these actives are moreover exclusively natural and are not the subject of chemical modifications.

The invention further relates to a cosmetic composition or pharmaceutical composition which comprises at least one extract described above.

Advantageously, the concentration of the extract is between 0.01 and 10% by weight of the total composition.

Advantageously, the extract is in a mixture with an excipient which is acceptable via the topical route, in particular a cosmetically or dermatologically acceptable excipient.

For these compositions, the excipient contains for example at least one compound selected from the group consisting of preservatives, emollients, emulsifiers, surfactants, moisturisers, thickeners, conditioners, matifying agents, stabilisers, antioxidants, texture agents, brightening agents, filmogenic agents, solubilisers, pigments, dyes, perfumes and solar filters. These excipients are preferably selected from the group consisting of amino acids and their derivatives, polyglycerols, esters, polymers and derivatives of cellulose, lanolin derivatives, phospholipids, lactoferrins, lactoperoxidases, sucrose-based stabilisers, E vitamins and its derivatives, natural and synthetic waxes, plant oils, triglycerides, insaponifiables, phytosterols, plant esters, silicones and its derivatives, protein hydrolysates, jojoba oil and its derivatives, lipo/hydrosoluble esters, betaines, aminoxides, plant extracts, esters of sucrose, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerol, sodium dihydroxycetyl, isopropyl hydroxycetyl ether, glycol stearate, triisononaoine, octyl cocoate, polyacrylamide, isoparaffin, laureth-7, a carbomer, propylene glycol, glycerol, bisabolol, dimethicone, sodium hydroxide, PEG 30-dipolyhydroxysterate, capric/caprylic triglycerides, cetearyl octanoate, dibutyl adipate, grape seed oil, jojoba oil, magnesium sulphate, EDTA, cyclomethicone, xanthan gum, citric acid, sodium lauryl sulphate, mineral waxes and oils, isostearyl isostearate, propylene glycol dipelargonate, propylene glycol isostearate, PEG 8 Beeswax, hydrogenated palm heart oil glycerides, hydrogenated palm oil glycerides, lanolin oil, sesame oil, cetyl lactate, lanolin alcohol, castor oil, titanium dioxide, lactose, sucrose, low density polyethylene, and an isotonic saline solution.

Advantageously, the compositions cited above are formulated in a form selected from the group consisting of a solution, which is aqueous or oily, an aqueous cream or gel or an oily gel, notably in a pot or in a tube, notably a shower gel, a shampoo, a milk, an emulsion, a microemulsion or a nanoemulsion, notably an oil-in-water or water-in-oil or multiple or silicone-containing microemulsion or nanoemulsion, a lotion, notably in a glass bottle, a plastic bottle or in a measure bottle or in an aerosol, an ampoule, a liquid soap, a dermatological bar, an ointment, a foam, an anhydrous product, preferably a liquid, pasty or solid anhydrous product, e.g. in the form of a stick, notably in the form of a lipstick.

The invention further relates to the use of at least one extract described above for the manufacture of a composition for increasing the concentration of active TGFb1 in the skin, notably in the dermis.

The invention further relates to the use of at least one extract described above for the manufacture of a composition for promoting the proliferation of fibroblasts.

The invention further relates to the use of at least one extract described above for the manufacture of a composition for increasing the synthesis of the constituents of the extracellular matrix, notably by increasing the synthesis of collagen, and/or increasing the synthesis of the protease inhibitors (TIMP), and/or increasing the synthesis of the proteoglycans and/or hyaluronic acid, and/or increasing the expression of the isoforms of fibronectin and/or the synthesis of the fibronectin receptors, and/or increasing the synthesis of elastin.

The invention further relates to the use of at least one extract described above for the manufacture of a composition for inhibiting the destructive activity of the extracellular proteins of the extracellular matrix.

The invention further relates to the use of at least one extract described above for the manufacture of a composition for exerting an anti-wrinkle or anti-ageing effect.

According to an embodiment of the present invention, the extract is prepared by extraction with a solvent. The solvent can be polar or not. Said solvent is preferably selected from the group consisting of pentane, decane, cyclohexane, hexane, petroleum ether, monochloromethane, dichloromethane, chloroform, isopropanol, propanol, ethyl acetate, ethanol, methanol, acetone, butylene glycol, propylene glycol, pentylene glycol, glycerol, water, and any mixture of at least two of these solvents, in particular hydro-alcoholic or hydro-glycolic mixtures.

Advantageously, the extract is purified. The extracts originate mainly from aqueous extraction, the MP diol and butylene glycol were used with the view to the affinity of some. The extracts of soya, of oats, of pea, of wheat, of maize, of quinoa and of pigeon bean or horse bean are extracted from the seed; on the other hand, the extracts of spring restharrow, of white mulberry tree, of yucca, of scutellaria and of parsley are extracted from the root, and finally, the extracts of tomato, of potato, mango, of date, of kiwi, of grapefruit, of papaya, of pineapple, of passion fruit, of apple and of dwarf palm are extracted from the fruit or from the berries. Silk (animal origin), the fish roe and the fish are extracted, dried and then treated.

Advantageously, the extract is diluted between 0.01% and 10% (w/w) in a solvent selected from water, glycols, including butylene glycol, a mixture of water and glycol and in particular butylene glycol, MP diol and ethanol.

Other aims, features and advantages of the invention will appear clearly to the person skilled in the art upon reading the explanatory description which makes reference to the following Examples which are given solely as an illustration and in no way limit the scope of the invention.

The Examples make up an integral part of the present invention and any feature appearing novel with respect to any prior state of the art from the description taken in its entirety, including the Examples, makes up an integral part of the invention in its function and in its generality.

Thus, every Example is of general scope.

Furthermore, in the Examples, all the percentages are given by weight, unless indicated otherwise, the temperature is expressed in degrees Celsius unless indicated otherwise, and the pressure is atmospheric pressure, unless indicated otherwise.

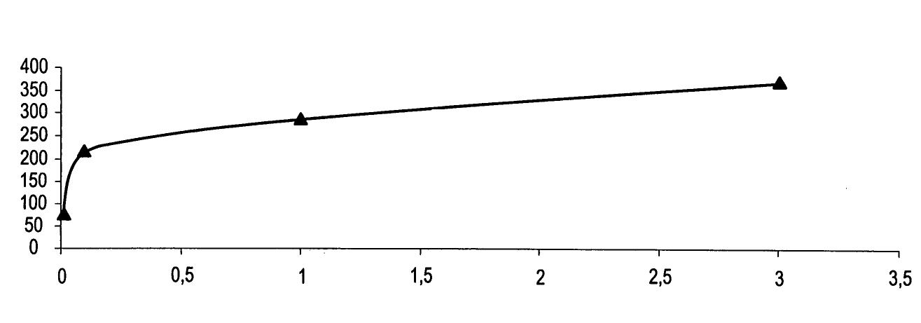

FIG. 1 represents the percentage of transcription of the elastin gene as a function of the concentration of the extract of dwarf palm, with reference to Example 31.

### EXAMPLES OF THE PRESENT INVENTION

### Example 1

### Extract of Soya

The protein fraction of soya is enriched from the soya seed by any physical or chemical procedure which enables such an enrichment. The protein fraction thus obtained is then dried by any conventional industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 2

### Extract of Oats

The protein fraction of oats (Avena sativa) is enriched from oat seeds by any physical or chemical procedure which enables such an enrichment. The protein fraction thus obtained is then dried by any conventional industrial process. 50 g of this product are then dissolved in a mixture made up of 550 g of demineralised water and 400 g of butylene glycol. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 3

### Extract of Fruits of Dwarf Palm

The lipophilic fraction (oils, sterols, waxes . . . ) of dwarf palm (Serenoa repens) is enriched from the fruits by any physical or chemical process which enables such an enrichment. Extraction by supercritical CO2 is preferred. The lipidic fraction thus obtained is separated from the insoluble fraction by filtration, centrifugation or any other method.

Example 3a: 10 g of this product are then dissolved in 990 g of butylene glycol. After 1 hour of mechanical agitation, the insoluble fraction is separated from the soluble fraction by filtration, centrifugation, ultra-filtration or any other method which enables this separation. The soluble fraction is used in the rest of the description of the invention.

Example 3b: 10 g of this product are then dissolved in 900 g of butylene glycol. After 1 hour of mechanical agitation, the insoluble fraction is separated from the soluble fraction by filtration, centrifugation, ultra-filtration or any other method which enables this separation. The soluble fraction is used in the rest of the description of the invention.

Example 3c: 300 g of this product are then dissolved in 700 g of butylene glycol. After 1 hour of mechanical agitation, the insoluble fraction is separated from the soluble fraction by filtration, centrifugation, ultra-filtration or any other method which enables this separation. The soluble fraction is used in the rest of the description of the invention.

### Example 4

### Extract of Silk

A protein hydrolysate of proteins of silk (Morus alba) is prepared by conventional methods of enzymatic or chemical hydrolysis. The protein fraction thus obtained is then dried by any conventional industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 5

### Extract of White Mulberry Tree

The root of white mulberry tree (Morus alba) is ground and then soaked in distilled water, and is then dried by any conventional industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 6

### Extract of Spring Restharrow

The root of spring restharrow (Ononis spinosa) is dried and then ground by any industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 7

### Extract of Pigeon Bean or Horse Bean

The protein fraction of pigeon bean or horse bean (Vicia faba) from the pigeon bean seed or horse bean seed by any physical or chemical process which enables such an enrichment. The protein fraction thus obtained is then dried by any conventional industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 8

### Extract of Tomato

The fruit of tomatoes (Solanum lycopersicum) is dried and then ground by any industrial process. 10 g of this product are then dissolved in 990 g of MP diol. After 1 hour of mechanical agitation, the insoluble fraction is separated from the soluble fraction by filtration, centrifugation, ultra-filtration or any other method which enables this separation. The soluble fraction is used in the rest of the description of the invention.

### Example 9

### Extract of Fish Roe

White fish roe is dried by any conventional means. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 10

### Protein Extract of Pea

The protein fraction of pea (Pisum sativum) is enriched from the pea seed by any physical or chemical process which enables such an enrichment. The protein fraction thus obtained is then dried by any conventional industrial process. 200 g of this product are then dissolved in 800 g of a mixture made up of 700 g of absolute ethanol and 300 g of demineralised water; the soluble extract is then dried by any conventional means and 30 g of this product are dispersed in 970 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 11

### Extract of Fish Flour

A suspension of fish flesh is prepared from white fish (ling, cod, haddock, etc . . . ) by any physical or chemical process which enables such a preparation. The fraction thus obtained is then dried by any conventional industrial process and 50 g of this product are dispersed in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 12

### Extract of Wheat Germ

Wheat germ is separated mechanically from the wheat seed (Triticum aestivum) and is reduced to a wheat germ powder by any physical or chemical process which enables such a grinding. The fraction thus obtained is then dried by any conventional industrial process. 30 g of this product are then dissolved in 970 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 13

### Extract of Potato

The protein fraction of potato (Solanum tuberosum) is enriched from the potato during the process of extraction of starch by any physical or chemical process which enables such an enrichment. The protein fraction thus obtained is then dried by any conventional industrial process. 100 g of this product are thus dispersed in 900 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 14

### Extract of Scutellaria

The roots of scutellaria (Scutellaria baicalensis) are extracted by enrichment in using an ethanolic solution in the hot (50-60° C.). After filtration of the insoluble matter by any conventional method, the ethanolic solution is concentrated and then dried by any conventional industrial process. 10 g of this product are then dissolved in 990 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 15

### Extract of Maize

The protein fraction of maize (Zea mays) is enriched from maize seed by any physical or chemical process which enables such an enrichment. The protein fraction thus obtained is then dried by any conventional industrial process. 30 g of this product are dispersed in 970 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 16

### Extract of Biotechnology-Modified Mango

A crude extract of mangoes (Mangifera indica) is prepared from the fruit by any physical or chemical process which enables the preparation of such an extract. The crude extract thus obtained is then dried by any conventional industrial process. 100 g of this product are then dissolved in 900 g of demineralised water, to which lactic ferments (Lactobacillus plantarum) are added. After 72 hours of fermentation, the insoluble fraction is separated from the soluble fraction by filtration, centrifugation, ultra-filtration or any other method which enables this separation. The soluble fraction is used in the rest of the description of the invention.

### Example 17

### Extract of Biotechnology-Modified Date

A crude extract of dates (Phoenix dactilifera) is prepared from the fruit by any physical or chemical process which enables the preparation of such an extract. The crude extract thus obtained is then dried by any conventional industrial process. 100 g of this product are then dissolved in 900 g of demineralised water, to which lactic ferments (Lactobacillus plantarum) are added. After 72 hours of fermentation, the insoluble fraction is separated from the soluble fraction by filtration, centrifugation, ultra-filtration or any other method which enables this separation. The soluble fraction is used in the rest of the description of the invention.

### Example 18

### Extract of Biotechnology-Modified Kiwi

A crude extract of kiwi (Actinidia chinensis) is prepared from the fruit by any physical or chemical process which enables the preparation of such an extract. The crude extract thus obtained is then dried by any conventional industrial process. 100 g of this product are then dissolved in 900 g of demineralised water, to which lactic ferments (Lactobacillus casei rhamnosus) are added. After 72 hours of fermentation, the insoluble fraction is separated from the soluble fraction by filtration, centrifugation, ultra-filtration or any other method which enables this separation. The soluble fraction is used in the rest of the description of the invention.

### Example 19

### Extract of Biotechnology-Modified Papaya

A crude extract of papaya (Carica papaya) is prepared from the fruit by any physical or chemical process which enables the preparation of such an extract. The crude extract thus obtained is the dried by any conventional industrial process. 100 g of this product are then dissolved in 900 g of demineralised water, to which beer yeast ferments (Saccharomyces cerevisiae) are added. After 72 hours of fermentation, the insoluble fraction is separated from the soluble fraction by filtration, centrifugation, ultra-filtration or any other method which enables this separation. The soluble fraction is used in the rest of the description of the invention.

### Example 20

### Extract of Biotechnology-Modified Apple

A crude extract of apple (Malus pumila) is prepared from the fruit by any physical or chemical process which enables the preparation of such an extract. The crude extract thus obtained is then dried by any conventional industrial process. 100 g of this product are then dissolved in 900 g of demineralised water, to which lactic ferments (Lactobacillus acidophilus) are added. After 72 hours of fermentation, the insoluble fraction is separated from the soluble fraction by filtration, centrifugation, ultra-filtration or any other method which enables this separation. The soluble fraction is used in the rest of the description of the invention.

### Example 21

### Extract of Quinoa Seed Flour

A crude extract of quinoa (Chenopodium quinoa) is prepared from the seeds by any physical or chemical process which enables the preparation of such an extract. The crude extract thus obtained is then dried by any conventional industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 22

### Extract of Parsley Root

Parsley roots (Petroselinum sativum) are dried and then finely ground by any industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 23

### Extract of Pineapple

A crude extract of pineapple (Ananas comosus) is prepared from the fruit by any physical or chemical process which enables the preparation of such an extract. The crude extract thus obtained is then dried by any conventional industrial process. 50 g of this product are then dissolved in 950 g of demineralised water. After 18 hours of mechanical agitation, the insoluble fraction is separated from the soluble fraction by filtration, centrifugation, ultra-filtration or any other method which enables this separation. The soluble fraction is used in the rest of the description of the invention.

### Example 24

### Extract of Biotechnology-Modified Passion Fruit

A crude extract of passion fruits (Passiflora edulis) is prepared from the fruit by any physical or chemical process which enables the preparation of such an extract. The crude extract thus obtained is then dried by any conventional industrial process. 100 g of this product are then dissolved in 900 g of demineralised water, to which lactic ferments (Lactobacillus plantarum) are added. After 72 hours of fermentation, the insoluble fraction is separated from the soluble fraction by filtration, centrifugation, ultra-filtration or any other method which enables this separation. The soluble fraction is used in the rest of the description of the invention.

### Example 25

### Extract of Yucca

A crude dry extract of yucca (Yucca schidigera) is prepared from the aerial part of the plant by any physical or chemical process which enables the preparation of such an extract. 10 g of this product are then dissolved in 990 g of butylene glycol. After 1 hour of mechanical agitation, the insoluble fraction is separated from the soluble fraction by filtration, centrifugation, ultra-filtration or any other method which enables this separation. The soluble fraction is used in the rest of the description of the invention.

### Example 26

### Extract of Grapefruit

A crude extract of grapefruit (Citrus grandis) is prepared from the fruit by any physical or chemical process which enables the preparation of such an extract. This product is then dissolved in a mixture made up of butylene glycol and water. After 1 hour of mechanical agitation, the insoluble fraction is separated from the soluble fraction by filtration, centrifugation, ultra-filtration or any other method which enables this separation. The soluble fraction is used in the rest of the description of the invention.

### Example 27

### Screening Test

A screening test which enables revealing the substances which are capable of activating this TGFb1-L was developed. Briefly, in order to evaluate the percentage of activated TGFb1-L, a determination via an active TGFb1 immunoenzymatic test is carried out. The experiments were carried out from human recombinant TGFb1. 40 μl of 1N HCl solution are added to 200 μl of a 0.1 μg/ml solution of TGFb1-L incubated for 18 hours at 4° C. without active. After homogenisation, the samples are incubated for 10 minutes at ambient temperature, and are then neutralised by adding 40 μl of 1.2N NaOH/1M HEPES solution.

The active TGFb1 content is evaluated by an ELISA technique which is described below. This value corresponds to a maximum amount of released TGFb1, this activation in acidic pH corresponding to a condition which is non-acceptable cosmetologically or dermopharmaceutically.

In a tube, 50 μl of the actives to be tested are added to 950 μl of 0.1 μg/ml TGFb1-L. After mixing, the samples are then incubated for 18 hours at 4° C. The screened extracts were tested at 5% in water.

Determination of activated TGFb1:

The active TGFb1 is then quantified in the reaction media with the aid of an ELISA test (Enzyme Linked ImmunoSorbent Assay), which is sensitive and specific to human active TGFb1. The type II TGFb1 receptor, to which TGFb1 binds, was pre-fixed onto a 96-well plate. The standards and the samples are then deposited in the wells and the TGFb1 present fixes to its immobilised receptor. After removal of non-bound substances by several rinses, a TGFb1-specific polyclonal antibody coupled to an enzyme is added into the wells. The excess antibodies are then removed and a solution containing a substrate of the enzyme is deposited in the wells. The conversion of this substrate by the enzyme generates a coloured product. The enzymatic reaction is stopped with the aid of a solution of sulphuric acid. The intensity of the colour obtained is measured by spectrophotometry at 450 nm and is proportional to the amount of TGFb1 activated by the sample tested.

The standard range thus enables calculating the concentration in pg/ml of activated TGFb1 in the reaction media as a function of the OD measured:
[TGFb1]=f(OD sample−OD control).

### Example 28

### Results According to the Screening of Example 27 of the Extracts Described in Examples 1 to 26

Here is the list of the best products tested at 5% in water:

| | LINNEUS | | Activated |
|---|---|---|---|
| NAME | NAME | NATURE | TGF |
| Extract of dwarf palm | Serenoa | dwarf palm | 3361.1 |
| berries | repens | berry | |
| Extract of fish roe | — | Fish roe | 1549.9 |
| Extract of soya | Glycine soja | Soya | 1299.9 |
| Extract of horse bean | Vicia faba | horse bean | 1247.9 |
| | equina | | |
| Extract of tomato | Solanum | Tomato | 1169.9 |
| | lycopersicum | | |
| Protein extract of pea | Pisum | Pea | 841.9 |
| | Sativum | | |
| Extract of fish flour | — | fish | 738.9 |
| Extract of wheat germ | Triticum | wheat | 735.9 |
| | Aestivum | | |
| Extract of mango/ | Mangifera | Mango | 624.9 |
| lactobacillus | indica | | |
| plantarum | | | |
| Extract of Date/ | Phoenix | Date | 568.9 |
| lactobacillus | dactilifera | | |
| plantarum | | | |
| Extract of Silk | Bombyx mori | Silk | 563.9 |
| Extract of Kiwi/ | Actinidia | Kiwi | 535.9 |
| lactobacillus casei | chinensis | | |
| rhamnosus | | | |
| Extract of potato | Solanum | potato | 498.6 |
| | tuberosum | | |
| Extract of Grapefruit | Citrus paradisi | Grapefruit | 477.4 |
| Extract of Papaya/ | Carica papaya | Papaya | 475.9 |
| saccaromyces | | | |
| cerevisiae | | | |
| Extract of pineapple | Ananas | pineapple | 353.9 |
| | comosus | | |
| Passion fruit/ | Passiflora | Passion fruit | 303.9 |
| lactobacillus | edulis | | |
| plantarum | | | |
| Extract of oats | Avena sativa | oats | 298.6 |
| Extract of Oughon | Scutellaria | scutellaria | 297.4 |
| | baicalensis | | |
| | georgi | | |
| Extract of sweetcorn | Zea mays | corn | 296.1 |
| Apple/lactobacillus | Malus pumila | apple | 237.9 |
| acidophilus | | | |
| Extract of quinoa | Chenopodium | Quinoa | 234.9 |
| | quinoa | | |
| Extract of Spring | Ononis spinosa | Spring | 229.9 |
| restharrow root | | restharrow root | |
| Extract of parsley root | Petroselinum | Parsley root | 184.3 |
| | sativum | | |
| Extract of Yucca | Yucca | Yucca | 171.1 |
| | schidigera | | |
| White mulberry tree | Morus alba | Mulberry root | 141.1 |
| | linne | | |
| Latent TGF (negative | | | 0 |
| control) | | | |

In the following Examples, the extract of dwarf palm is obtained according to Example 3.

### Example 29

### Dose Effect of a Product of the Invention: The Extract of Dwarf Palm

| | Average of the | |
|---|---|---|
| % of extract | concentrations of | |
| of Serenoa | activated TGFb1 | Standard |
| repens | (pg/ml) | deviation |
| 0.1 | 2258.1 | 9.6 |
| 1 | 1516.3 | 239.8 |
| 3 | 1174.9 | 127 |
| 5 | 709.5 | 17 |
| 10 | 302.6 | 24.8 |

### Example 30

### Effect of the Extract of Dwarf Palm Upon the Synthesis of Fibronectin

The study of the synthesis of fibronectin comprises three steps activating TGFb1-L, incubating activated TGFb1-L on monolayer normal human dermis fibroblasts, and determining fibronectin synthesised from the culture medium.

The experiments were carried out from human recombinant TGFb1-L. The TGFb1-L solution is stored at a concentration of 8 μg/ml. A dilution to the eighth of the TGFb1-L at 8 μg/ml in PBS 1× is made in order to test the activation of TGFb1-L at 1 μg/ml. 10 μl of the extract of dwarf palm are added to 990 μl of TGFb1-L at 1 μg/ml. After agitation, the product is placed in incubation for 18 hours at 4° C.

The solution of activated TGFb1 is then diluted to the tenth in culture medium without FBM serum (Promocell-Germany). Experiment controls are prepared: two negative controls, including an FBM control and a TGFb1-L control; and two positive active TGFb1 controls (Sigma-France) at 10 ng/ml and at 1 ng/ml diluted in the FBM. Each solution is deposited at the rate of 2 ml/well on monolayer cultures of confluent fibroblasts in 6-well plates. The cultures are incubated for 48 hours at 37° C. in an atmosphere containing 5% of CO2. The fibronectin is then quantified in the culture media with the aid of an EIA test (Enzyme Immuno Assay), which is sensitive and specific to human fibronectin. The fibronectin receptor, to which the fibronectin is bound, was pre-fixed onto a 96-well plate. The standards and the samples are then deposited in the wells and the fibronectin present fixes to its immobilised receptor. After removal of the non-bound substances by several rinses, a fibronectin-specific polyclonal antibody coupled to an enzyme is added into the wells. The excess of antibodies is then removed and a solution containing a substrate of the enzyme is deposited in the wells. The enzymatic reaction is stopped with the aid of a solution of sulphuric acid. The intensity of the colour obtained measured by spectrophotometry at 450 nm is proportional to the amount of fibronectin synthesised. The results are expressed as a percentage with respect to the negative TGFb1-L control. In observing the results, it can be considered that the TGFb1-L control stimulates the synthesis of fibronectin, and this can be explained by the capacity that the fibroblasts have to activate the TGFb1-L. This parameter is got rid of by comparing the amount of fibronectin synthesised by the cells in the presence of TGFb1-L activated by the active, to the amount of fibronectin synthesised by the cells in the presence of TGFb1-L.

| | Average % stimulation | Standard deviation |
|---|---|---|
| Extract of dwarf palm | 21.1 | 10.6 |
| Active TGFb1 10 ng/ml | 30.4 | 6.9 |
| Active TGFb1 1 ng/ml | 12.4 | 8.7 |

### Example 31

### Dose Effect of the Extract of Dwarf Palm on the Transcription of the Gene Encoding Elastin

The study of the transcription of the gene encoding elastin comprises three steps: activating TGFb1-L, incubating activated TGFb1 on monolayer normal human dermis fibroblasts, and determining the RNAs (ribonucleic acids) extracted from the cell mat by RT-PCR (Retro Transcriptase-Polymerase Chain Reaction).

The experiments were carried out from human recombinant TGFb1-L. The solution of TGFb1-L is stored at a concentration of 8 μg/ml. A dilution to the eighth of the TGFb1-L at 8 μg/ml in PBS 1× is carried out in order to test the activation of TGFb1-L at 1 μg/ml. In a tube, 1 ml of TGFb1-L at 1 μg/ml is added to a necessary volume of extract of dwarf palm, which enables testing the following concentrations: 0.01%, 0.1%, 1% and 3%. The mixture is incubated for 18 hours at 4° C., and the solution of activated TGFb1-L is then diluted in culture medium without FBM serum (Promocell-Germany). Experiment controls are prepared: two negative controls, including an FBM control and a TGFb1-L control, and one positive active TGFb1 control (Sigma-France) at 1 ng/ml diluted in the FBM. Each solution is deposited at the rate of 1 ml/well on monolayer cultures of confluent fibroblasts in 24-well plates. The cultures are incubated for 24 hours at 37° C. in an atmosphere containing 5% of CO2. After removal of the culture medium and rinses of the cell mats, the total RNAs are extracted from the cells. The extraction is carried out by lysing the cells on a positively charged silica column. The negatively charged RNA is therefore retained on the column and is then eluted into a 96-well plate. The quantification and the purity of the RNAs extracted is done by reading on the spectrophotometer at 260/280 nm. The solutions of RNA are standardised in order to be finally at 5 ng/ml, and are then aliquoted into a 96 PCR plate by 10 μl/well in counting one plate for the gene analysed: elastin and one plate for the housekeeping gene, actin. The RT-PCR determination enables amplifying the RNAs of the elastin gene compared to the reference gene: actin. This determination is done with the aid of a Quantitech Sybergreen RT-PCR kit (Qiagen-France) and of the specific primers of the amplified genes. The programme is made up of a step of activation of reverse transcriptase (30 minutes at 50° C.), of a step of denaturation of reverse transcriptase and of the activation of polymerase (15 minutes at 95° C.) and of 50 cycles which comprise the opening of the strands (15 seconds at 95° C.), the fixing of the primers (30 seconds at 60° C.) and the action of polymerase (30 seconds at 72° C.). The results obtained (FIG. 1) correspond to a cycle number read for a fluorescence of 0.01.

FIG. 1 represents the percentage of transcription of the elastin gene as a function of the concentration of the extract of dwarf palm. The axis of the abscissas represents the tested concentration of the extract of dwarf palm (percentage by weight), and the axis of the ordinates represents the percentage by weight of transcription of the elastin gene.

A ratio for each sample is calculated between the cycle number read for the elastin and the cycle number read for actin.

The extract of dwarf palm at 0.1% doubles the transcription of the elastin gene, at 1% increases it 2.8 times, and finally at 3% increases it 3.7 times. A dose effect is therefore noted: the more the concentration of the extract of dwarf palm increases, the more the transcription of the gene increases, and significantly.

### Example 32

### Effect of the Extract of Dwarf Palm on the Synthesis of Collagen

This experiment was carried out in order to evaluate the effects of the TGFb1-L, activated or not, on the incorporation of 3H-proline in the neo-synthesized proteins. The study of the synthesis of collagen comprises three steps: activating TGFb1-L, incubating the activated or non-activated TGFb1-L on the monolayer normal human dermis fibroblasts, and incorporating proline and analysing the radioactivity incorporated.

The experiments were carried out from human recombinant TGFb1-L used at concentrations of 10 ng/ml, 100 ng/ml and 1000 ng/ml. In a tube, 10 μl of the extract of dwarf palm are added to 990 μl of each solution of TGFb1-L. After mixing, the samples are then incubated for 18 hours at 4° C. The solution of activated TGFb1-L is then diluted to the 1/10^{th} in DMEM culture medium (Invitrogen-France) with 1% FCS (foetal calf serum). Experiment controls are prepared: two negative controls, including a DMEM control and a TGFb1-L control; and two positive controls, including vitamin C at 20 μg/ml and active TGFb1 (Sigma-France) at 10 ng/ml diluted in the DMEM. Each solution is placed in contact with monolayer cultures of confluent normal human dermis fibroblasts in 96-well plates. The cultures are incubated for 72 hours at 37° C. in an atmosphere containing 5% of CO2. The last 24 hours of incubation are done in the presence of the label 3H-proline (Amersham Biosciences-France) at 42 Ci/mmol.

The analysis of the radioactivity incorporated is done by precipitation with TCA (trichloracetic acid), which is collected on a filter, rinses of the filtrate with TCA and 70° ethanol, and finally by counting in liquid scintillation.

The extract of dwarf palm has increased the effect of the TGFb1-L on the fraction of the proteins deposited. This effect is visible in the presence of TGFb1-L at 100 and 1,000 ng/ml.

| | | % stimulation with | |
|---|---|---|---|
| | Concentration of | respect to the | Standard |
| | TGFb1-L (ng/ml) | TGFb1-L | deviation |
| Active TGF | — | 35.3 | 9.2 |
| 10 ng/ml | | | |
| 1% of extract of | 100 | 68.3 | 14.8 |
| dwarf palm | 1000 | 36.5 | 6.7 |

### Example 33

### The Extract of Dwarf Palm is Still Capable of Activating the TGFb1-L After Transcutaneous Penetration

The study of the transcutaneous penetration comprises three steps: the transcutaneous penetration of the extract of dwarf palm, the activation of TGFb1-L by the permeate, and the determination of the activated TGFb1-L.

The transcutaneous permeation experiment consists of inserting a rat skin biopsy between the donor and receiver compartments of a Franz cell. One gram of extract of dwarf palm is applied onto the rat skin, the receiver containing PBS buffer. A control cell was prepared without active. The transcutaneous penetration of the active is evaluated over 24 hours, after which time the permeate containing the extract of dwarf palm is recovered. This permeate is then evaluated for its capacity to activate the TGFb1-L. For this, 40 μl of 1N HCl solution are added to 200 μl of a solution of TGFb1-L at 0.1 μg/ml incubated for 18 hours at 4° C. without active. After homogenisation, the samples are incubated for 10 minutes at ambient temperature, and are then neutralised by adding 40 μl of 1.2 N NaOH/1M HEPES solution.

The content of active TGFb1 is evaluated by an ELISA technique described above. This value corresponds to a maximum amount of released TGFb1, this activation in acidic pH corresponding to a condition which is non-acceptable cosmetologically or dermopharmaceutically.

In a tube, 100 μl of the permeate are added to 900 μl of TGFb1-L at 0.1 μg/ml. After mixing, the product is incubated for 18 hours at 4° C. The active TGFb1 is then quantified in the reaction media with the aid of an ELISA test (Enzyme Linked ImmunoSorbent Assay), which is sensitive and specific to human active TGFb1. The type II TGFb1 receptor, to which TGFb1 binds, was pre-fixed onto a 96-well plate. The standards and the samples are then deposited in the wells and the TGFb1 present fixes to its immobilised receptor. After removal of non-bound substances by several rinses, a TGFb1-specific polyclonal antibody coupled to an enzyme is added to the wells. The excess antibodies are then removed and a solution containing a substrate of the enzyme is deposited in the wells. The conversion of this substrate by the enzyme generates a coloured product. The enzymatic reaction is stopped with the aid of a solution of sulphuric acid. The intensity of the colour obtained measured by spectrophotometry at 450 nm is proportional to the amount of TGFb1 activated by the sample tested.

The standard range thus enables calculating the concentration in pg/ml of activated TGFb1 in the reaction media as a function of the OD measured:
[TGFb1]=f(OD sample−OD control).

| | Average of the activated | |
|---|---|---|
| | TGFb1-L concentration | |
| | (pg/ml) | Standard deviation |
| Permeate of control cell | 164.4 | — |
| Permeate of the cell | 1013.8 | 211 |
| treated with the extract of | | |
| dwarf palm | | |

### Examples 34

### Use of the Products of the Invention in Oil-In-Water Emulsion Type Cosmetic or Pharmaceutical Formulations

Formulation 34a:

| A | Water | qsp 100 |
|---|---|---|
| | Butylene Glycol | 2 |
| | Glycerine | 3 |
| | Sodium Dihydroxycetyl- | 2 |
| | phosphate Isopropyl | |
| | hydroxycetyl Ether | |
| B | Glycol Stearate SE | 14 |
| | Triisononaoin | 5 |
| | Octyl Cocoate | 6 |
| C | Butylene Glycol, | 2 |
| | Methylparaben, | |
| | Ethylparaben, Propylparaben, | |
| | pH adjusted to 5.5 | |
| D | Products of the invention | 0.01-10% |

Formulation 34b:

| A | Water | qsp 100 |
|---|---|---|
| | Butylene Glycol | 2 |
| | Glycerine | 3 |
| | Polyacrylamide, Isoparaffin, | 2.8 |
| | Laureth-7 | |
| B | Butylene Glycol, | 2 |
| | Methylparaben, | |
| | Ethylparaben, Propylparaben; | |
| | Phenoxyethanol, | 2 |
| | Methylparaben, | |
| | Propylparaben, Butylparaben, | |
| | Ethylparaben | |
| | Butylene Glycol | 0.5 |
| D | Products of the invention | 0.01-10% |

Formulation 34c:

| A | water | qsp 100 |
|---|---|---|
| | Carbomer | 0.50 |
| | Propylene Glycol | 3 |
| | Glycerol | 5 |
| B | Octyl Cocoate | 5 |
| | Bisabolol | 0.30 |
| | Dimethicone | 0.30 |
| C | Sodium Hydroxide | 1.60 |
| D | Phenoxyethanol, | 0.50 |
| | Methylparaben, | |
| | Propylparaben, Butylparaben, | |
| | Ethylparaben | |
| E | Perfume | 0.30 |
| F | Products of the invention | 0.01-10% |

### Example 35

### Use of the Products of the Invention in a Water-In-Oil Type Formulation

| | | |
|---|---|---|
| A | PEG 30 dipolyhydroxystearate | 3 |
| | Capric Triglycerides | 3 |
| | Cetearyl Octanoate | 4 |
| | Dibutyl Adipate | 3 |
| | Grape Seed Oil | 1.5 |
| | Jojoba Oil | 1.5 |
| | Phenoxyethanol, | 0.5 |
| | Methylparaben, | |
| | Propylparaben, Butylparaben, | |
| | Ethylparaben | |
| B | water | qsp 100 |
| | Glycerine | 3 |
| | Butylene Glycol | 3 |
| | Magnesium Sulphate | 0.5 |
| | EDTA | 0.05 |
| C | Cyclomethicone | 1 |
| | Dimethicone | 1 |
| D | Perfume | 0.3 |
| E | Products of the invention | 0.01-10% |

### Example 36

### Use of the Products of the Invention in a Shampoo or Shower Gel Type Formulation

| A | water | qsp 100 |
|---|---|---|
| | Xanthan Gum | 0.8 |
| B | Butylene Glycol, | 0.5 |
| | Methylparaben, | |
| | Ethylparaben, Propylparaben | |
| | Phenoxyethanol, | 0.5 |
| | Methylparaben, | |
| | Propylparaben, Butylparaben, | |
| | Ethylparaben | |
| C | Citric acid | 0.8 |
| D | Sodium Laureth Sulphate | 40.0 |
| E | Product of the invention | 0.01-10% |

### Example 37

### Use of the Products of the Invention in a Lipstick Type Formulation and Other Anhydrous Products

| | | |
|---|---|---|
| A | Mineral Wax | 17.0 |
| | Isostearyl Isostearate | 31.5 |
| | Propylene Glycol Dipelargonate | 2.6 |
| | Propylene Glycol Isostearate | 1.7 |
| | PEG 8 Beeswax | 3.0 |
| | Hydrogenated Palm Kernel Oil | 3.4 |
| | Glycerides, | |
| | Hydrogenated Palm Glycerides | |
| | Lanolin Oil | 3.4 |
| | Sesame Oil | 1.7 |
| | Cetyl Lactate | 1.7 |
| | Mineral Oil, Lanolin Alcohol | 3.0 |
| B | Castor Oil | qsp 100 |
| | Titanium Dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | 0.01-5% |

### Example 38

### Use of the Products of the Invention in an Aqueous Gels Formulation (Eyeliners, Slimmers, etc . . . )

| A | Water | qsp 100 |
|---|---|---|
| | Carbomer | 0.5 |
| | Butylene Glycol | 15 |
| | Phenoxyethanol, Methylparaben, | 0.5 |
| | Propylparaben, Butylparaben, | |
| | Ethylparaben | |
| B | Products of the invention | 0.01-10% |

### Example 39

### Evaluation of the Cosmetic Acceptation of a Preparation Containing the Subject of the Invention

Toxicology tests were carried out on the compound obtained according to Example 2 incorporated at 10% in a 0.5% xanthan gel, by an ocular evaluation in the rabbit, by the study of the absence of abnormal toxicity by single oral administration in the rat and by the study of the sensitising power in the guinea pig.

Evaluation of the Primary Irritation of the Skin in the Rabbit:

The preparations described above are applied without dilution at the dose of 0.5 ml on the skin of 3 rabbits according to the method recommended by the OECD in relation to the study of “the acute irritant/corrosive effect on the skin”.

The products are classed according to the criteria defined in the Decision of Feb. 1, 1982 published in the Official Journal of the French Republic (the “JORF”) of Feb. 2, 1982.

The results of this test have enabled concluding that the preparations can be considered as being non-irritant for the skin, in the sense of Directive 91/326 EEC used pure or without dilution.

Evaluation of the Ocular Irritation in the Rabbit:

The preparations described above were instilled pure and in one batch at the rate of 0.1 ml in the eye of three rabbits according to the method recommended by the directive of the OECD No. 405 of Feb. 24, 1987, in relation to the study of “the acute irritant/corrosive effect on the eyes”. The results of this test enable concluding that the preparations can be considered as non-irritant for the eyes, in the sense of Directive 91/326 EEC used pure or without dilution.

Test on the Absence of Abnormal Toxicity by Single Oral Administration in the Rat:

The preparations described were administered in one batch orally at the dose of 2 g/Kg of body weight, to 5 male rats and 5 female rats according to a protocol inspired from the Directive of the OECD No. 401 of Feb. 24, 1987 and adapted to cosmetic products.

The LD0 and LD50 are found to be greater than 2,000 mg/Kg. The preparations tested are therefore not classed amongst the preparations which are dangerous by ingestion.

Evaluation of the Skin Sensitisation Potential in the Guinea Pig:

The preparations described are subjected to the maximization test described by Magnusson and Kligmann, a protocol which is in agreement with the directive line No. 406 of the OECD.

The preparations are classed as non-sensitising by contact with the skin.

1. A method selected from the group consisting of a method for increasing the concentration of active TGFb1 in the skin, of a method for promoting the proliferation of fibroblasts in the skin, of a method for increasing the synthesis of the constituents of the extracellular matrix (ECM) in the skin, a method for inhibiting degradation of the extracellular proteins of the extracellular matrix in the skin, and a method for exerting an anti-wrinkle or anti-ageing effect of the skin, said method comprising administering at least one skin acceptable natural extract which converts Latent TGFbeta-1 (TGFb1-L) into active TGFbeta-1 (active TGFb1).

2. The method of claim 1, wherein the extract which converts latent TGFbeta-1 (TGFb1-L) into active TGFbeta-1 (active TGFb1) cleaves the bond which unites the TGFb1 to the protein LAP (latency associated protein).

3. The method of claim 1, wherein the concentration of active TGFb1 is increased in the dermis.

4. The method of claim 1, wherein the increase of synthesis of the constituents of the ECM is selected from the group consisting of increasing the synthesis of collagen, increasing the synthesis of the protease inhibitors (TIMP), increasing the synthesis of the proteoglycans, increasing the synthesis of hyaluronic acid, increasing the expression of the isoforms of fibronectin, increasing the synthesis of the fibronectin receptors, increasing the synthesis of elastin, and any combination thereof.

5. The method of claim 1, wherein the extract is selected from the group consisting of an extract of soya (Glycine soja), an extract of oats (Avena sativa), an extract of dwarf palm (Serenoa repens), an extract of silk, an extract of roots of white mulberry tree (Morus alba), an extract of Spring restharrow (Ononis spinosa), an extract of pigeon bean or horse bean (Vicia faba equina), an extract of tomato (Solanum lycopersicum), an extract of fish roe, an protein extract of pea (Pisum sativum), fish flour, wheat germ (Triticum aestivum), an extract of mango (Mangifera indica), an extract of date (Phoenix dactilifera), an extract of kiwi (Actinidia chinensis), an extract of potato (Solanum tuberosum), an extract of grapefruit (Citrus paradisi), an extract of papaya (Carica papaya), an extract of pineapple (Ananas comosus), an extract of passion fruit (Passiflora edulis), an extract of scutellaria (Scutellaria baicalensis), an extract of maize (Zea mays), an extract of apple (Malus pumila), an extract of quinoa flour (Chenopodium quinoa), an extract of parsley root (Petroselinum sativum), an extract of yucca (Yucca schidigera), and any one of the mixtures thereof.

6. The method of claim 5, wherein said natural extract has been submitted to a fermentation step with bacteria.

7. The method of claim 1, wherein the concentration of the natural extract is ranging between 0.01 and 10% by weight of the total composition.

8. The method of claim 1, wherein said natural extract is an extract of soya.

9. The method of claim 1, wherein said natural extract comprises an extract of oat.

10. The method of claim 9, wherein said extract of oat is an extract of oat seeds of Avena sativa.

11. The method of claim 1, wherein said natural extract is an extract of fruits of dwarf palm.

12. The method of claim 11, wherein said extract of fruits of dwarf palm is a lipophilic extract of dwarf palm.

13. The method of claim 12, wherein said lipophilic extract is a lipophilic extract of dwarf palm Serenoa repens.

14. The method of claim 1, wherein said natural extract comprises a water soluble fraction of a protein hydrolysate of proteins of silk.

15. The method of claim 14, wherein said silk is Morus alba.

16. The method of claim 1, wherein said natural extract is a water soluble fraction of the roots of white mulberry tree.

17. The method of claim 1, wherein said natural extract is a water soluble fraction of the roots of spring restharrow.

18. The method of claim 1, wherein the natural extract is a water soluble fraction of the protein fraction of pigeon bean seeds or horse bean seeds.

19. The method of claim 1, wherein the natural extract is a water soluble fraction of the fruit of tomato.

20. The method of claim 1, wherein said natural extract is a water soluble fraction of the roots of white mulberry tree.

21. The method of claim 1, wherein said natural extract is a water soluble fraction of white fish roe.

22. The method of claim 1, wherein the natural extract is a water soluble fraction of the protein fraction of pea seeds.

23. The method of claim 1, wherein said natural extract is a water soluble fraction of fish flour or flesh prepared from white fish.

24. The method of claim 23, wherein said white fish is selected from ling, code haddock.

25. The method of claim 1, wherein said natural extract is a water soluble fraction of wheat germ.

26. The method of claim 1, wherein the natural extract is an extract of a water soluble fraction of the protein fraction of maize seeds.

27. The method of claim 1, wherein the natural extract is a water soluble fraction of the fermentation product obtained after fermentation with a lactic ferment of mango.

28. The method of claim 1, wherein the natural extract is a water soluble fraction of the fermentation product obtained after fermentation with a lactic ferment of dates.

29. The method of claim 1, wherein the natural extract is a water soluble fraction of the fermentation product obtained after fermentation with a lactic ferment of kiwi.

30. The method of claim 1, wherein the natural extract is a water soluble fraction of the fermentation product obtained after fermentation with a lactic ferment of papaya.

31. The method of claim 1, wherein the natural extract is a water soluble fraction of the fermentation product obtained after fermentation with a lactic ferment of apple.

32. The method of claim 1, wherein the natural extract is a water soluble fraction of quinoa seeds.

33. The method of claim 1, wherein the natural extract is a water soluble fraction of parsley roots.

34. The method of claim 1, wherein the natural extract is a water soluble fraction of a crude extract of pineapple.

35. The method of claim 1, wherein the natural extract is a water soluble fraction of the fermentation product obtained after fermentation with a lactic ferment of passion fruits.

36. The method of claim 1, wherein the natural extract is a butylene glycol soluble fraction of yucca.

37. The method of claim 1, wherein the natural extract is a butylene glycol soluble fraction of a crude extract of grapefruits.

38. A composition which comprises at least one cosmetically or dermatologically acceptable natural extract converting TGFb1-L into active TGFb1 under cosmetically or pharmaceutically acceptable conditions, and a cosmetically or pharmaceutically acceptable excipient.

39. A topical cosmetical or dermatological composition which comprises at least one cosmetically or dermatologically acceptable natural extract converting TGFb1-L into active TGFb1 under cosmetically or pharmaceutically acceptable conditions; and a cosmetically or dermatologically acceptable excipient.

40. The composition of claim 39, wherein the extract is selected from the group consisting of an extract of soya (Glycine soja), an extract of oats (Avena sativa), an extract of dwarf palm (Serenoa repens), an extract of silk, an extract of roots of white mulberry tree (Morus alba), an extract of Spring restharrow (Ononis spinosa), an extract of pigeon bean or horse bean (Vicia faba equina), an extract of tomato (Solanum lycopersicum), an extract of fish roe, an protein extract of pea (Pisum sativum), fish flour, wheat germ (Triticum aestivum), an extract of mango (Mangifera indica), an extract of date (Phoenix dactilifera), an extract of kiwi (Actinidia chinensis), an extract of potato (Solanum tuberosum), an extract of grapefruit (Citrus paradisi), an extract of papaya (Carica papaya), an extract of pineapple (Ananas comosus), an extract of passion fruit (Passiflora edulis), an extract of scutellaria (Scutellaria baicalensis), an extract of maize (Zea mays), an extract of apple (Malus pumila), an extract of quinoa flour (Chenopodium quinoa), an extract of parsley root (Petroselinum sativum), an extract of yucca (Yucca schidigera), and any one of the mixtures thereof.

41. The composition of claim 39, wherein the natural extract is a water soluble fraction of the fermentation product obtained after fermentation with bacteries of an extract selected from the group consisting of an extract of soya (Glycine soja), an extract of oats (Avena sativa), an extract of dwarf palm (Serenoa repens), an extract of silk, an extract of roots of white mulberry tree (Morus alba), an extract of Spring restharrow (Ononis spinosa), an extract of pigeon bean or horse bean (Vicia faba equina), an extract of tomato (Solanum lycopersicum), an extract of fish roe, an protein extract of pea (Pisum sativum), fish flour, wheat germ (Triticum aestivum), an extract of mango (Mangifera indica), an extract of date (Phoenix dactilifera), an extract of kiwi (Actinidia chinensis), an extract of potato (Solanum tuberosum), an extract of grapefruit (Citrus paradisi), an extract of papaya (Carica papaya), an extract of pineapple (Ananas comosus), an extract of passion fruit (Passiflora edulis), an extract of scutellaria (Scutellaria baicalensis), an extract of maize (Zea mays), an extract of apple (Malus pumila), an extract of quinoa flour (Chenopodium quinoa), an extract of parsley root (Petroselinum sativum), an extract of yucca (Yucca schidigera), and any one of the mixtures of these extracts.

42. The composition of claim 39, wherein the concentration of the cosmetically or dermatologically acceptable natural extract is ranging between 0.01 and 10% by weight of the total composition.
